(19) United States
(12) Patent Application Publication
(10) Pub. No.: US 2008/0194679 A1
(43) Pub. Date: Aug. 14, 2008

(54) Compositions and methods for increasing metabolic activity in animal tissue

(76) Inventors: H. DeWayne Ashmead, Fruit Heights, UT (US); Stephen D. Ashmead, West Haven, UT (US)

Correspondence Address: THORPE NORTH & WESTERN, LLP., P.O. Box 1219, SANDY, UT, 84091-1219, US

(21) Appl. No.: 11/706,869

(22) Filed: Feb. 14, 2007

(51) Int. Cl. A61K 31/28; A61P 39/04; C07F 19/00

(52) U.S. Cl. 514/492; 556/1

(57) ABSTRACT

The present invention is directed to methods and compositions which include high nitrogen metal amino acid chelates that can increase the metabolic activity or metal concentration in animals. In one embodiment, an amino acid composition can comprise an amino acid chelate with a first metal and first amino acid ligand, where the first amino acid ligand is devoid of a disulfide bond and has at least two nitrogen atoms, and an amino acid complex different from the amino acid chelate having a second metal and second amino acid ligand. The amino acid composition can also include nitrogen salts, proteinates, urea, nitric acid, carnitine, creatine, glucosamine, chondroitin, chitosan, nitrogen-containing botanicals, and combinations thereof.

### FIELD OF THE INVENTION

The present invention is drawn to methods and compositions with high nitrogen-containing chelates. Additionally, the present invention is drawn to the use of high nitrogen-containing amino acid chelates for increasing and/or retaining nitrogen and metal content within an animal tissue for enhancing metabolic activity.

### BACKGROUND OF THE INVENTION

Amino acid chelates are generally produced by the reaction between α-amino acids and metal ions having a valence of two or more to form a ring structure. In such a reaction, the positive electrical charge of the metal ion can be neutralized by the electrons available through the carboxylate or free amino groups of the α-amino acid.

Traditionally, the term “chelate” has been loosely defined as a combination of a metallic ion bonded to one or more ligands to form a heterocyclic ring structure. Under this definition, chelate formation through neutralization of the positive charge(s) of the metal ion may be through the formation of ionic, covalent or coordinate covalent bonding. An alternative and more modern definition of the term “chelate” requires that the metal ion be bonded to the ligand solely by coordinate covalent bonds forming a heterocyclic ring. In either case, both are definitions that describe a metal ion and a ligand forming a heterocyclic ring.

Chelation can be confirmed and differentiated from mixtures of components or more ionic complexes by infrared spectra through comparison of the stretching of bonds or shifting of absorption caused by bond formation. As applied in the field of mineral nutrition, there are certain “chelated” products that are commercially utilized. The first is referred to as a “metal proteinate.” The American Association of Feed Control officials (AAFCO) has defined a “metal proteinate” as the product resulting from the chelation of a soluble salt with amino acids and/or partially hydrolyzed protein. Such products are referred to as the specific metal proteinate, e.g., copper proteinate, zinc proteinate, etc. Sometimes, metal proteinates are erroneously referred to as “amino acid” chelates.

The second product, referred to as an “amino acid chelate,” when properly formed, is a stable product having one or more five-membered rings formed by a reaction between the amino acid and the metal. The American Association of Feed Control Officials (AAFCO) has also issued a definition for metal amino acid chelates. It is officially defined as the product resulting from the reaction of a metal ion from a soluble metal salt with amino acids having a mole ratio of one mole of metal to one to three (preferably two) moles of amino acids to form coordinate covalent bonds. The average weight of the hydrolyzed amino acids must be approximately 150 and the resulting molecular weight of the chelate must not exceed 800. The products are identified by the specific metal forming the chelate, e.g., iron amino acid chelate, copper amino acid chelate, etc.

In further detail with respect to amino acid chelates, the carboxyl oxygen and the α-amino group of the amino acid each bond with the metal ion. Such a five-membered ring is defined by the metal atom, the carboxyl oxygen, the carbonyl carbon, the α-carbon and the α-amino nitrogen. The actual structure will depend upon the ligand to metal mole ratio and whether the carboxyl oxygen forms a coordinate covalent bond or an ionic bond with the metal ion. Generally, the ligand to metal molar ratio is at least 1:1 and is preferably 2:1 or 3:1. However, in certain instances, the ratio may be 4:1. Most typically, an amino acid chelate with a divalent metal can be represented at a ligand to metal molar ratio of 2:1 according to Formula 1 as follows:

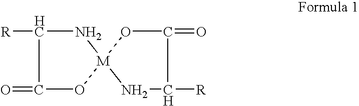

In the above formula, the dashed lines represent coordinate covalent bonds, covalent bonds, or ionic bonds. Further, when R is H, the amino acid is glycine, which is the simplest of the α-amino acids. However, R could be representative of any other side chain that, when taken in combination with the rest of the ligand structure(s), results in any of the other twenty or so naturally occurring amino acids used in protein synthesis. All of the amino acids have the same configuration for the positioning of the carboxyl oxygen and the α-amino nitrogen with respect to the metal ion. In other words, the chelate ring is defined by the same atoms in each instance, even though the R side chain group may vary.

With respect to both amino acid chelates and proteinates, the reason a metal atom can accept bonds over and above the oxidation state of the metal is due to the nature of chelation. For example, at the α-amino group of an amino acid, the nitrogen contributes to both electrons used in the bonding. These electrons fill available spaces in the d-orbitals of the metal ion forming a coordinate covalent bond. Thus, a metal ion with a normal valency of +2 can be bonded by four bonds when fully chelated. In this state, the chelate is completely satisfied by the bonding electrons and the charge on the metal atom (as well as on the overall molecule) is zero. As stated previously, it is possible that the metal ion can be bonded to the carboxyloxygen by either coordinate covalent bonds or ionic bonds. However, the metal ion is preferably bonded to the α-amino group by coordinate covalent bonds only.

The structure, chemistry, bioavailability, and various applications of amino acid chelates are well documented in the literature, e.g. Ashmead et al., Chelated Mineral Nutrition, (1982), Chas. C. Thomas Publishers, Springfield, III.; Ashmead et al., Intestinal Absorption of Metal Ions, (1985), Chas. C. Thomas Publishers, Springfield, III.; U.S. Pat. Nos. 4,020,158; 4,167,564; 4,216,143; 4,216,144; 4,599,152; 4,725,427; 4,774,089; 4,830,716; 4,863,898; 5,292,538; 5,292,729; 5,516,925; 5,596,016; 5,882,685; 6,159,530; 6,166,071; 6,207,204; 6,294,207; and 6,614,553; each of which are incorporated herein by reference.

One advantage of amino acid chelates in the field of mineral nutrition is attributed to the fact that these chelates are readily absorbed from the gut and into mucosal cells by means of active transport. In other words, the minerals can be absorbed along with the amino acids as a single unit utilizing the amino acid(s) as a carrier molecule. Therefore, the problems associated with the competition of ions for active sites and the suppression of specific nutritive mineral elements by others can be avoided.

As such, metal amino acid chelates have been used as a dietary supplement for a variety of nutritional metals and amino acids. Even though chelation generally offers better mineral absorbability, absorption is a complex biological function influenced by many variables. As such, methods and complexes with improved absorption characteristics and that provide increased health benefits continue to be sought through ongoing research and development efforts.

### SUMMARY OF THE INVENTION

Briefly, and in general terms, the invention is directed to methods and compositions that are formulated such that metal amino acid chelates which are present can increase the metabolic activity and metal tissue concentration in an animal. In one embodiment, an amino acid composition can comprise an amino acid chelate with a first metal and first amino acid ligand, wherein the first amino acid ligand is devoid of a disulfide bond and has at least two nitrogen atoms. The composition can further include an amino acid complex which is different from the amino acid chelate, and which includes a second metal and second amino acid ligand.

In another embodiment, an amino acid chelate composition can comprise a first metal, an amino acid ligand having at least two nitrogen atoms, and a second nitrogen-containing compound selected from proteinates, urea, nitrates, carnitine, creatine, glucosamine, chondroitin, chitosan, nitrogen-containing botanicals, and combinations thereof.

Additionally, a method of increasing a metabolic activity in an animal tissue can comprise administering an amino acid chelate including a multi-nitrogen-containing amino acid ligand and a metal to an animal in an amount sufficient to i) raise the nitrogen and the metal concentration within the tissue, ii) retain the metal content in the tissue for a greater period of time compared to when delivered as a compound with less nitrogen, and iii) enhance metabolic activity of the tissue.

Other embodiments will also be described herein which illustrate, by way of example, features of the present invention.

### DETAILED DESCRIPTION OF THE PREFERRED EMBODIMENT(S)

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It is noted that, as used in this specification and the appended claims, the singular forms “a,” “an,” and, “the” include plural referents unless the context clearly dictates otherwise. Thus, for example, reference to “a chelate” can include one or more of such chelates, reference to “an amount of nitrogen” can include reference to one or more amounts of nitrogen, and reference to “the amino acid” can include reference to one or more amino acids.

As used herein, the term “naturally occurring amino acid” or “traditional amino acid” shall mean amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

As used herein, the term “high nitrogen” or “high nitrogen-containing” or “multi-nitrogen-containing” refers to any compound that contains at least 2 nitrogen atoms. These terms are meant to include chelates, ligands, or other compounds, molecules, or complexes.

As used herein, the term “nitrogen-containing” refers to any compound, molecule, complex, or chelate that contains a nitrogen atom.

As used herein, the term “amino acid chelate” refers to both the traditional definitions and the more modern definition of chelate as cited previously. Specifically, with respect to chelates that utilize traditional amino acid ligands, i.e., those used in forming proteins, chelate is meant to include metal ions bonded to proteinaceous ligands forming heterocyclic rings. Between the carboxyl oxygen and the metal, the bond can covalent or ionic, but is preferably coordinate covalent. Additionally, at the α-amino group, the bond is typically a coordinate covalent bond. Proteinates of naturally occurring amino acids are included in this definition.

As used herein, the term “metal” refers to nutritionally relevant metals including divalent and trivalent metals that can be used as part of a nutritional supplement, are known to be beneficial to humans, and are substantially non-toxic when administered in traditional amounts, as is known in the art. Examples of such metals include copper, zinc, manganese, iron, chromium, calcium, potassium, sodium, magnesium, cobalt, nickel, molybdenum, vanadium, strontium, selenium, and the like. This term also includes nutritional semi-metals including, but not limited to, silicon.

As used herein, the term “proteinate” when referring to a metal proteinate is meant to include compounds where the metal is chelated or complexed to hydrolyzed or partially hydrolyzed protein forming a heterocyclic ring. Coordinate covalent bonds, covalent bonds, and/or ionic bonds may be present between the metal and the proteinaceous ligand of the chelate or chelate/complex structure. As used herein, proteinates are included when referring to amino acid chelates. However, when a proteinate is specifically mentioned, it does not include all types of amino acid chelates, as it only includes those with hydrolyzed or partially hydrolyzed protein.

As used herein, the term “amino acid chelate” and “metal amino acid chelate” are used interchangeable, as by definition, a chelate requires the presence of a metal.

As used herein, the term “carnitine” refers to the compound having the following structure, including both L and D forms:

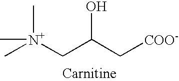

Carnitine can be a ligand for use in accordance with the present invention. In this configuration, the molecule is said to be “zwitterionic,” because of two full opposite charges carried by the molecule. A somewhat unique characteristic of carnitine comes from the fact that it exists in this zwittwerionic form, regardless of pH. All of the naturally occurring or traditional amino acids can form zwitterions, but the ionization of most amino acids is dependent on the pH of the solution in which they are dissolved. Carnitine typically exists as a zwitterion independent of the solution pH. Carnitine chelates or carnitine complexes are specific types of carnitine, and thus, are included in the definition of carnitine.

As used herein, the term “glandular substance” refers to any of the various animal organs or tissues that synthesize substances needed by the body and release them through ducts or directly into the bloodstream. For example, this term includes animal organs such as, but not limited to, pituitary, brain, heart, pancreas, hypothalamus, and liver. Glandular substances can be ground or otherwise formulated for coadministration with chelates in accordance with certain embodiments of the present invention. Complexes and other compounds that include glandular substances are included in the definition of glandular substances.

As used herein, the term “chitosan” refers to a linear polysaccharide composed of randomly distributed β-(1-4)-linked D-glucosamine (deacetylated unit) and N-acetyl-D-glucosamine (acetylated unit). Chitosan is readily available from a number of commercial sources. Complexes and other compounds that include chitosan are included in the definition of chitosan.

As used herein, the term “chondroitin” includes compounds such as chondroitin sulfate, which is a sulfated glycosaminoglycan (GAG) composed of a chain of alternating sugars (N-acetyl-galactosamine and glucuronic acid). It is usually found attached to proteins as part of a proteoglycan. A chondroitin chain can have over 100 individual sugars, each of which can be sulfated in variable positions and quantities. Complexes and other compounds that include chondroitin are included in the definition of chondroitin.

As used herein, the term “creatine” refers to a nitrogenous organic acid that naturally occurs in vertebrates and helps to supply energy to muscle cells, generally having the following structure:

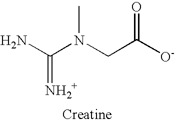

It is noted that creatine chelates, creatine monohydrate, creatine complexes, or the like are specific types of creatine, and thus, are included in the definition of creatine.

As used herein, the term “glucosamine” refers to an amino sugar that is a precursor in the biochemical synthesis of glycosylated proteins and lipids. This term includes the various D and L forms, having the general structure, C₆H₁₄NO₅. The α-D form is shown below:

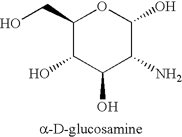

Complexes and other compounds that include glucosamine are included in the definition of glucosamine.

As used herein, the term “nitrogen-containing botanical extracts” refers to any botanical extract that contains at least one nitrogen atom. Examples of nitrogen-containing botanical extract agents that may be used with the methods and compositions of the present invention include, without limitation, extracts from the following: Ginseng, Ginko Biloba, Dong Quai, Hawthorn berry, St. John's Wort, Saw Palmetto, Kava (Piper methysticum), Rose Hips, Echinacea, Licorice Root, Grape seed, Chammomile, Hempseed, Aloe Vera, Cordyceps, Ho Shou Wu, Dandelion, Gynostemma, mushrooms, Notginseng, Dan Shen, Noni, Garlic, Nopal, Milk Thistle, Causena Lansium, Crocus Sativus, Danshen (saliva miltiorrhize), Dongui (Radix angelicae sinesis), Eucommia, Evening primrose, Gastrodia elata, Hops, Mishmi bitter (coptis sinesis), Morning star (Uncaria rhychophylla), Passion flower, Physostigmine, Securinega, Suffructicosa, Scutellaria baicalensis, Siberian cork tree (phellodendron amurense), Skullcap, Valerian, astragalus, coriolus versicolor, ginger, rhodiola rosea, German chamomile, Green tea (Camellia Sinensis), Horn goat weed (epimedium sagittatum), and mixtures thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

With these definitions in mind, high nitrogen-containing metal amino acid chelates can increase metabolic activity in an animal as well as mineral adsorption in an animal tissue. Generally, chelation has been shown to increase the absorbability of minerals since they are readily absorbed from the gut and into mucosal cells by means of active transport. In other words, the minerals are often absorbed along with the amino acids as a single unit, thereby utilizing the amino acids as carrier molecules. This being stated, it has been found that high nitrogen-containing metal amino acid chelates have an unexpected effect on the metabolic activity of various animals. Generally, the high nitrogen-containing metal amino acid chelates can increase the mineral concentration in the animal tissue and retain the metal in the tissue for a longer period of time. For example, in mammals, e.g., cows, sows, poultry, etc., metabolic activity such as milk production, weight gain, fertility, feed conversion, etc. can be increased by such administration more so than by delivering metal compounds with less nitrogen. Additionally, such increased metabolic activity can provide an increased quantity and quality of associated products, such as, but not limited to, milk products and/or meat. Furthermore, the increased metabolic activity can reduce morbidity and mortality.

In one embodiment, an amino acid chelate composition can comprise a metal amino acid chelate and an amino acid complex. The amino acid chelate can include a first metal and first amino acid ligand devoid of a disulfide bond and having at least two nitrogen atoms. The amino acid complex can contain a second metal and a second amino acid ligand, such that the complex is different than the chelate. The difference between the chelate and the complex can be due to the differences in the metals, ligands, or chemical structure, where the structures can have a different spatial orientation or different bonding types. In one embodiment, the metals are different and the amino acid ligands are the same. In another embodiment, the metals are the same while the amino acids are different. In still another embodiment, both the metals and the ligands are different. It is noted that in one embodiment, the amino acid complex can be a second amino acid chelate.

Generally, the amino acid chelate composition can include a high nitrogen amino acid chelate as well as other compounds. The high nitrogen amino acid chelates can include amino acid ligands such as, but not limited to, arginine, asparagine, glutamine, histidine, lysine, ornithine, and tryptophan, including dipeptides, tripeptides, and tetrapeptides thereof. Specifically, a high nitrogen amino acid chelate contains at least one amino acid ligand that has at least two nitrogen atoms. In one embodiment, a high nitrogen amino acid chelate contains at least one amino acid ligand that has at least three nitrogen atoms. In another embodiment, a high nitrogen amino acid chelate contains at least one amino acid ligand that has at least four nitrogen atoms.

Additionally, the amino acid complex can be a non-chelated complex, or alternatively, a second amino acid chelate. In one embodiment, the second amino acid chelate or non-chelated complex can include an amino acid ligand such as, but not limited to, alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof. In another embodiment, the second amino acid chelate or non-chelate complex can contain at least one amino acid ligand having at least two nitrogen atoms.

The metals contemplated for use in the compositions and methods of the present invention are generally nutritionally relevant metals, as defined previously. Specific examples include, but are not limited to, copper, zinc, manganese, iron, chromium, calcium, sodium, silicon, potassium, magnesium, cobalt, nickel, molybdenum, vanadium, selenium, strontium, and the like. It is noted that certain metals may perform better for certain targeted metabolic activity. For example, if the desire is to enhance general growth, metals such as zinc, iron, or calcium may be preferable for use in the amino acid chelate and/or the amino acid complex (which may optionally also be a chelate). If the desire is to enhance milk production, metals such as manganese, zinc, calcium, or copper may be preferable for use in the amino acid chelate and/or the amino acid complex (which may also optionally also be a chelate). If the desire is to enhance reproduction, metals such as zinc or manganese may be preferable for use in the amino acid chelate and/or the amino acid complex (which may also optionally also be a chelate). Other metabolic activities and metal choices may be determined by one skilled in the art. If the desire is to reduce infant mortality, iron may be preferably for use in the amino acid chelate and/or the amino acid complex (which may also optionally also be a chelate). Generally, the methods and compositions can be formulated for any animal, e.g., humans, mammals, fowl, fish, crustacean, etc.

An amino acid chelate composition can include numerous combinations of metals to ligands in the form of chelates and other compounds and complexes. Such arrangements are contemplated by the present invention and may be manufactured through generally known preparative complex and/or chelation methods. It is not the purpose of the present invention to describe how to prepare amino acid chelates that can be used with the present invention. Suitable methods for preparing such amino acid chelates can include those described in U.S. Pat. Nos. 4,830,716 and/or 5,516,925, to name a few. However, combinations of such chelates as part of a composition for increasing metabolic activity or increasing and retaining nitrogen and metal content in a tissue are included as an embodiment of the present invention. In one embodiment, the first amino acid chelate and the second amino acid complex each have an amino acid ligand to metal ratio from about 1:1 to about 4:1. In another embodiment, the amino acid chelate composition has an amino acid chelate to amino acid complex ratio from about 10:1 to about 1:10, by weight.

In this and other embodiments, the amino acid chelate composition can include an additional third nitrogen-containing compound. The third nitrogen-containing compound can be, but is not limited to, proteinates, urea, nitrates, carnitine, creatine, glucosamine, chondroitin, chitosan, nitrogen-containing botanicals, glandular substances, and combinations thereof. The amino acid chelate composition can also include a third compound that preferably is also a nitrogen-containing compound, e.g., chelate, complex, or salt, including a third metal and an anion or coordination ligand. The third metal can be any metal as previously defined. The third metal may be the same or different than the first and/or second metals. Specifically, all three metals may be the same, all may be different, or 2 metals may be the same with the third metal being different. The anion or coordination ligand can be, for example, another amino acid chelate or complex; proteins; peptides, polypeptides; amino acid sulfates; nitrates; cyano-compounds; soy isolate or soy protein; feather meal; albumin; casein; urea; whey; gelatin; gluten; or ammonium compounds. In each of these embodiments, the third compound can also be a nitrogen-containing compound including 1 nitrogen atom, 2 nitrogen atoms, 3 nitrogen atoms, or even 4 nitrogen atoms.

In an alternative embodiment, an amino acid chelate composition can comprise a high nitrogen-containing amino acid chelate and a second nitrogen-containing compound including, but not limited to, proteinates, urea, nitric acid, carnitine, creatine, glucosamine, chondroitin, chitosan, nitrogen-containing botanicals, and combinations thereof. In one embodiment, the second nitrogen-containing compound can include at least one nitrogen atom, but can also include at least two nitrogen atoms. In one embodiment, the amino acid chelate composition can further include a glandular substance. As described previously, the high nitrogen-containing amino acid chelate can include at least one amino acid ligand and a metal. Further, as previously defined, the metal can be copper, zinc, manganese, iron, chromium, calcium, potassium, sodium, silicon, magnesium, cobalt, nickel, molybdenum, vanadium, strontium, selenium, or the like, and metal choice can be based, in part, on the target metabolic activity of the animal. The high nitrogen amino acid chelate can have amino acid ligands such as, but not limited to, arginine, asparagine, glutamine, histidine, lysine, ornithine, and tryptophan, including dipeptides, tripeptides, and tetrapeptides thereof. Specifically, a high nitrogen amino acid chelate contains at least one amino acid ligand that has at least two nitrogen atoms. In one embodiment, the amino acid ligand includes at least three nitrogen atoms. In another embodiment, the amino acid ligand includes at least four nitrogen atoms.

Also as previously described, the amino acid chelate composition can have an amino acid to metal ratio from about 1:1 to about 4:1, and/or the amino acid chelate composition can have an amino acid chelate to second nitrogen-containing compound weight ratio from about 10:1 to about 1:10. The amino acid chelate composition can further include a nitrogen-containing salt including a second metal and an anion. The second metal can be copper, zinc, manganese, iron, chromium, calcium, potassium, sodium, silicon, magnesium, cobalt, nickel, molybdenum, vanadium, strontium, selenium, or the like, and metal choice can be based, in part, on the target metabolic activity of the animal and/or the other metal(s) used the composition. The anion or coordination ligand can be, for example, nitrates, amino acid sulfates, or ureates.

The present invention also provides a method of increasing a metabolic activity in animal tissue by administering an amino acid chelate composition containing at least one multi-nitrogen-containing amino acid chelate having at least one multi-nitrogen-containing amino acid ligand and a metal to an animal in an amount sufficient to i) raise the nitrogen and the metal concentration within the tissue, ii) retain the metal content in the tissue for a greater period of time compared to when delivered as a compound with less nitrogen, and iii) enhance metabolic activity of the tissue. The multi-nitrogen-containing amino acid ligand generally includes at least 2 nitrogen atoms and can be, but is not limited to, arginine, asparagine, cystine, glutamine, histidine, lysine, ornithine, and tryptophan, including dipeptides, tripeptides, and tetrapeptides thereof. In one embodiment, the multi-nitrogen-containing amino acid ligand can have at least three nitrogen atoms. In another embodiment, the multi-nitrogen-containing amino acid ligand can have at least four nitrogen atoms. The metal can be any metal as previously defined including, but not limited to, copper, zinc, manganese, iron, chromium, calcium, potassium, sodium, silicon, magnesium, cobalt, nickel, molybdenum, vanadium, strontium, selenium, or the like.

The present method can include co-administration of a second amino acid complex (which can be a chelate) that is different than the multi-nitrogen-containing amino acid chelate, where the second amino acid chelate can have a second metal and at least one second amino acid ligand. The second amino acid ligand can be any amino acid including, but not limited to, alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof. In one embodiment, the second amino acid ligand has at least two nitrogen atoms.

The second metal can also be any metal as previously defined including, but not limited to copper, zinc, manganese, iron, chromium, calcium, potassium, sodium, silicon, magnesium, cobalt, nickel, molybdenum, vanadium, strontium, selenium, and the like, and can be selected for a specific metabolic activity increase. The metal and second metal may be the same or different. Also, the multi-nitrogen-containing amino acid ligand and the second amino acid ligand can be the same or different. The present method includes the various combinations that may be obtained by these ligands and metals as defined; i.e., different metals with the same ligands, different metals with different ligands, the same metals with different ligands, etc.

Additionally, the present combination of a multi-nitrogen-containing amino acid chelate with a second amino acid complex may have the same metals and ligands but may have a different chemical connectivity such that the chelates are still considered different for the purposes of the present invention. For example, differences can be related to different spatial orientations or bond types, e.g., complex vs. chelate, ligand to metal ratios, etc. The multi-nitrogen-containing amino acid chelate and second amino acid chelate or complex can each have an amino acid ligand to metal ratio from about 1:1 to about 4:1. The amino acid chelate composition can have a multi-nitrogen-containing amino acid chelate to second amino acid chelate weight ratio from about 10:1 to about 1:10.

In each of the above-described embodiments, the compositions and methods of the present invention can provide nitrogen content to an animal from about 0.5 wt % to about 35 wt %, based on the composition as a whole. Additionally, the compositions and methods of the present invention can provide nitrogen content to an animal from about 5 wt % to about 35 wt %, based solely on the nitrogen-containing compounds found in the composition. Also as previously mentioned, the metabolic activity can enhance milk production, weight gain, enhanced growth, enhanced fertility, reduced morbidity, reduced tissue fat, or enhanced feed conversion. The compositions can be formulated for parenteral delivery. The compositions for administration can have formulations including oral, injection, powder, tablet, capsule, gel, liquid, or paste. In one embodiment, the formulation is oral or injection.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### EXAMPLES

The following provides examples of high nitrogen amino acid compositions in accordance with the compositions and methods previously disclosed. Additionally, some of the examples include studies performed showing the effects of high nitrogen metal amino acid chelates on animals in accordance with embodiments of the present invention.

### Example 1

To about 700 ml of deionized water containing 50 grams citric acid is added 616 grams of arginine to form a clear solution. To this solution of citric acid and arginine is slowly added 55.8 grams of elemental iron. The solution is heated at about 50° C. for 48 hours, or until substantially all the iron is observed to go into solution. The product is cooled, filtered, and dried yielding a ferric trisarginate amino acid chelate.

Calcium creatine chelate having a 1:1 ligand to metal molar ratio is prepared, first, by combining the following ingredients: 540.00 ml of water at 50 to 55° C.; 150.00 grams of creatine monohydrate; 59.98 grams of calcium oxide; and 23.43 grams of 85% o-phosphoric acid. The reaction mixture is heated to about 50° C. to 55° C. and spray providing a calcium creatine powder.

A high nitrogen amino acid composition is obtained by dissolving 53 grams of iron trisarginate powder and 17.3 grams of calcium creatine powder in 500 ml of water, heating to about 50° C. to 55° C., and drying, forming a powder having an iron to calcium molar ratio of about 1:1 and an arginine to creatine molar ratio of about 3:1.

### Example 2

A copper carbonate solution is prepared by adding 6.1 parts by weight of cupric carbonate to 80.9 parts by weight water. This solution is allowed to stand without agitation for about two hours. To this solution is added 16.4 parts by weight of asparagine, and the mixture is slowly stirred for about two more hours. To the hazy solution is added 65 parts by weight of a 15 wt % citric acid solution and the mixture is stirred until a clear solution is observed. This solution is dried resulting in a copper bisasparaginate powder.

A solution is prepared including 10.1 parts by weight of histidine dissolved in 82.2 parts by weight water containing 1.0 part by weight sodium carbonate. To this solution is added 4.4 parts by weight zinc oxide. The molar ratio of histidine to zinc is 2:1. The reaction mixture is allowed to stand for about 14 hours and turned an opalescent color. After standing, the mixture is heated to about 70° C. and is dried to obtain a zinc bishistidinate amino acid chelate powder.

A high nitrogen amino acid chelate composition is obtained by dry blending 73 grams of the copper bisasparaginate with 85 grams of zinc bishistidinate amino acid chelate to provide a homogenous amino acid composition with a copper to zinc molar ratio of about 1:1, and an asparagine to histidine molar ratio of about 1:1.

### Example 3

A mixture of 42.93 grams of zinc sulfate, 67 grams of lysine, and 30 grams of glycine are reacted in an aqueous environment for 60 minutes at a temperature of about 65 to 70° C. The reaction of the zinc sulfate, lysine, and glycine produces a zinc amino acid chelate having a ligand component to metal molar ratio of about 2:1, and a lysine to glycine molar ratio of about 1:1. Zinc nitrate is admixed with the zinc amino acid chelate in a 1:1 molar ratio providing a high-nitrogen zinc chelate/salt formulation.

### Example 4

To about 700 ml of deionized water containing 50 g citric acid is added 616 g of arginine to form a clear solution. To this solution of citric acid and arginine is slowly added 55.8 g of elemental iron. The solution is heated at about 50° C. for 48 hours, or until substantially all the iron is observed to go into solution. The product is cooled, filtered, and dried yielding a ferric trisarginate amino acid chelate.

About 250 grams of glycine was dissolved into 937.8 grams of water. Once the glycine was significantly dissolved, about 95 grams of calcium oxide was added. The solution was continually stirred for about 15 minutes until all of the calcium was dissolved. The reaction mixture is heated to about 50 to 55° C. and dried providing a calcium bisglycinate powder.

A high nitrogen amino acid composition is obtained by dissolving 106 grams of iron trisarginate powder and 31.2 grams of calcium bisglycinate powder in 500 ml of water, heating to about 50 to 55° C., and drying, forming a powder having an iron to calcium molar ratio of about 1:1 and an arginine to glycine molar ratio of about 3:2.

### Example 5

A high nitrogen amino acid chelate composition is prepared by the process as described in Example 2. A glandular substance/high nitrogen amino acid composition is formed by dry blending 50 grams of the copper bisasparaginate/zinc bishistidinate composition with 150 grams of liver powder, which is suitable for targeting liver, spleen, and bone marrow tissue in a mammal providing increased blood production.

### Example 6

A high nitrogen amino acid chelate composition is prepared by the process as described in Example 4. A creatine/high nitrogen amino acid chelate composition is further obtained by blending 50 grams of the iron trisarginate/calcium bisglycinate powder with 9.6 grams of creatine powder, providing an iron to calcium to creatine ratio of about 1:1:1.

### Example 7

A high nitrogen amino acid chelate composition is prepared by the process as described in Example 4. A carnitine/high nitrogen amino acid chelate composition is further obtained by blending 50 grams of the iron trisarginate/calcium bisglycinate powder with 75 grams of carnitine powder, providing an iron to calcium to carnitine ratio of about 1:1:1.

### Example 8

In an isotope study, three groups of zinc sufficient adult male rats (6/group) received a single intravenous (I.V.) dose of 0.06 mg zinc containing 10 microcurines of zinc chelated to either arginine or glycine or as zinc chloride (ZnCl₂). The arginine contained about twice as much nitrogen bonded to the zinc atom as did glycine. The ZnCl₂ had no nitrogen bonded to the zinc. Twenty four hours post-dosing, all animals were sacrificed and their testes, epididymis, and seminal vesicles were assayed for zinc. The male sex organs were targeted for these zinc assays because of the dominant role zinc plays in their proper functioning. In theory, the uptake of zinc from the three sources into the tissues should have been equivalent since all three sources were administered by I.V. and none of the animals was zinc deficient.

As seen in Table 1, however, tissue retention of either amino acid chelate source of zinc was significantly greater (p<0.05) than tissue retention of zinc from the IM source. This study also indicates that tissue retention of zinc is partially dependent on nitrogen being bonded to the metal. As noted above, arginine has about twice as much nitrogen in the ligand as glycine. Thus more zinc from the arginine chelate was retained in the tissues than from the glycine chelate and both chelates resulted in better zinc retention than did ZnCl₂.

TABLE 1
Deposition of ⁶⁵zinc from three sources into various tissue sites

| | ⁶⁵Zn Arginine | ⁶⁵Zn Glycine | ⁶⁵Zn Chloride |
|---|---|---|---|
| | (corrected | (corrected | (corrected |
| | counts/minute/mg | counts/minute/mg | counts/minute/mg |
| Tissue | of tissue) | of tissue) | of tissue) |
| Testes | 1.26 | 0.96 | Negligible |
| Epididymis | 1.03 | 0.73 | Negligible |
| Seminal | 2.53 | 1.98 | Negligible |
| Vesicle | | | |

### Example 9

Non-fasted rats were first anesthetized and then their intestines exposed. A 10 cm section of the ileum was tied off at both ends extending 15 cm to 5 cm proximal to the ileoceal valve. At each end, a 0.5 cm incision was made allowing the tied off section to be thoroughly washed with Ringers solution via the incisions. A solution containing ⁶⁵ZnCl₂ or ⁶⁵Zn Histidine (1×10⁻⁴M) was introduced into the tied off section. At 2 hours post dosing, the rats were killed by cervical dislocation. Their livers, hearts, and spleens were taken, weighed, dissected, and assayed by liquid scintillation for ⁶⁵Zn. The results are shown in the Table 2, as follows:

TABLE 2

| | Tissue treated with | Tissue treated with |
|---|---|---|
| | ⁶⁵Zn Histidine Solution | ⁶⁵Zn Chloride Solution |
| Organ | (number × 10⁻¹⁰ Zn/gm) | (number × 10⁻¹⁰ Zn/gm) |
| Liver | 13.0 ± 8.83 | 8.67 ± 5.67 |
| Heart | 1.67 ± 0.50 | 1.00 ± 0.33 |
| Spleen | 4.17 ± 0.67 | 3.50 ± 0.25 |

Note the greater tissue retention of the zinc when it was chelated to histidine. The high nitrogen-containing zinc amino acid chelate provided increased zinc concentration in each organ.

### Example 10

Ferrous iron was chelated to various high nitrogen-containing amino acid chelates. Six grams of such a chelate formula (0.6 g Fe) was fed daily to gestating sows beginning 4 weeks before expected farrowing. Another group of gestating sows received 2 g daily of ferrous fumarate (0.67 g Fe) for the same period. One week prior to farrowing, the dosages of iron were doubled for each group.

At farrowing blood samples were taken from each piglet and assayed for hemoglobin, hematocrit, and serum ferritin. The hemoglobin and hematocrit represent current iron status but serum ferritin represents iron storage for future use. The following table summarizes the results:

TABLE 3

| | Ferrous Amino Acid Chelate | Ferrous Fumarate |
|---|---|---|
| Hemoglobin (g/dL) | 9.6 | 9.7 |
| Hematocrit (%) | 32.4 | 33.2 |
| Serum Ferritin | 157.2^{a} | 20.1^{b} |
| (ng/ml) | | |

^{a/b}P < 0.01

Ferrous fumarate is reported to be well absorbed, being equivalent to ferrous sulfate, ferrous glycine sulfate, ferrous glutamate, and ferrous gluconate. High absorption of the iron from either source is obvious in the above study when one looks at either the hemoglobin or the hematocrit; however, the serum ferritin reflects the difference in tissue storage of the iron from the two sources. Ferrous fumarate contains no nitrogen, whereas the amino acid chelate includes high nitrogen-containing amino acids chelated to the iron.

### Example 11

A group of one day old chicks were administered 40 mg zinc per day per kg feed for 21 consecutive days. The sources of zinc were zinc nitrate (24.3% Zn), zinc glycinate (1:1) (26.2% Zn), zinc bisglycinate (2:1) (22.3% Zn), and zinc bisarginate (2:1) (15.7% Zn). At the end of 21 days, all chicks in all groups were killed, freeze dried, and a whole body assay for zinc performed. Results of the assay are reported in Tables 4 and 5 below:

TABLE 4
21 Day zinc retention on Whole Body Analysis

| | x̅ |
|---|---|
| Zn Nitrate | 13.241 mg Zn/chick |
| Zn Glycinate (1:1) | 13.424 mg Zn/chick |
| Zn Bisglycinate (2:1) | 14.282 mg Zn/chick |
| Zn Bisarginate (2:1) | 19.116 mg Zn/chick |

Zinc nitrate and zinc glycinate (1:1) contains the same amount of nitrogen in the molecule. Note zinc retention in the body is similar. Zinc bisglycinate (2:1) contains twice as much nitrogen as either zinc nitrate or zinc glycinate (1:1). Zinc retention increased to some degree. When zinc bisarginate (2:1) was administered, zinc retention increased over zinc bisglycinate (2:1) significantly. Zinc bisarginate (2:1) has twice as much nitrogen as zinc bisglycinate (2:1).

The average 21 day body weight and the gain per feed are shown in Table 5, as follows:

TABLE 5
Body Weight and Gain/Feed

| | | | | Zn |
|---|---|---|---|---|
| | Zn | Zn Glycinate | Zn Bisglycinate | Bisarginate |
| 21 Day | Nitrate | (1:1) | (2:1) | (2:1) |
| Body Weight (g) | 734.98 | 817.08 | 812.55 | 829.15 |
| Gain/Feed (g) | 0.74 | 0.77 | .078 | .078 |

The greater weight gains came with the increased zinc retention in the bodies of the chicks (facilitated by the higher nitrogen content) showing that greater metal retention leads to greater metabolic activity.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An amino acid chelate composition formulated for animal delivery, comprising:
(a) an amino acid chelate including a first metal and a first amino acid ligand, said first amino acid ligand being devoid of a disulfide bond and having at least two nitrogen atoms; and
(b) an amino acid complex including a second metal and a second amino acid ligand, wherein the amino acid complex is different than the amino acid chelate.

2. The amino acid chelate of claim 1, wherein the first amino acid ligand is different than the second amino acid ligand.

3. The amino acid chelate of claim 1, wherein the first metal is different than the second metal.

4. The amino acid chelate composition of claim 1, wherein the amino acid complex is also an amino acid chelate.

5. The amino acid chelate composition of claim 1, wherein the first amino acid ligand includes an amino acid selected from the group consisting of arginine, asparagine, glutamine, histidine, lysine, ornithine, and tryptophan, including dipeptides, tripeptides, and tetrapeptides thereof.

6. The amino acid chelate composition of claim 1, wherein the second amino acid ligand includes an amino acid selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

7. The amino acid chelate composition of claim 1, wherein the first and second metals are independently selected from the group consisting of copper, zinc, manganese, iron, chromium, calcium, potassium, sodium, silicon, magnesium, cobalt, nickel, molybdenum, vanadium, strontium, and selenium.

8. The amino acid chelate composition of claim 1, wherein the first and second metals are the same, and the first and second amino acid ligands are different.

9. The amino acid chelate composition of claim 1, wherein the first and second metals are different, and the amino acid ligands are different.

10. The amino acid chelate composition of claim 1, wherein the first and second metals are different, and the first and second amino acid ligands are the same.

11. The amino acid chelate composition of claim 1, wherein the first amino acid ligand includes at least three nitrogen atoms.

12. The amino acid chelate composition of claim 1, wherein the first amino acid ligand includes at least four nitrogen atoms.

13. The amino acid chelate composition of claim 1, wherein the second amino acid ligand includes at least two nitrogen atoms.

14. The amino acid chelate composition of claim 1, wherein the first amino acid chelate and the second amino acid complex each have an amino acid ligand to metal ratio from about 1:1 to about 4:1.

15. The amino acid chelate composition of claim 1, wherein the amino acid chelate composition has a first amino acid chelate to second amino acid complex ratio from about 10:1 to about 1:10.

16. The amino acid chelate composition of claim 1, further comprising a third nitrogen-containing compound.

17. The amino acid chelate composition of claim 16, wherein the third nitrogen-containing compound includes at least two nitrogen atoms.

18. The amino acid chelate composition of claim 16, wherein the third nitrogen-containing compound is selected from the group consisting of nitrogen-containing salts, proteinates, urea, nitric acid, carnitine, creatine, glucosamine, chondroitin, chitosan, nitrogen-containing botanicals, glandular substances, and combinations thereof.

19. The amino acid chelate composition of claim 1, wherein the nitrogen content from the amino acid chelate and amino acid complex is from about 5 wt % to about 35 wt %.

20. The amino acid chelate composition of claim 1, wherein the animal is a mammal.

21. The amino acid chelate composition of claim 1, wherein the animal is a human.

22. The amino acid chelate composition of claim 1, wherein the animal is a fowl.

23. The amino acid chelate composition of claim 1, wherein the animal is a fish.

24. The amino acid chelate composition of claim 1, wherein the animal is a crustacean.

25. The amino acid chelate composition of claim 1, wherein the amino acid complex is a metal bisglycinate chelate.

26. The amino acid chelate composition of claim 1, in oral dosage form.

27. An amino acid chelate composition formulated for animal delivery, comprising:
(a) an amino acid chelate including a metal and an amino acid ligand having at least two nitrogen atoms; and
(b) a second nitrogen-containing compound selected from the group consisting of proteinates, urea, nitric acid, carnitine, creatine, glucosamine, chondroitin, chitosan, nitrogen-containing botanicals, and combinations thereof.

28. The amino acid chelate composition of claim 27, wherein the metal is independently selected from the group consisting of copper, zinc, manganese, iron, chromium, calcium, potassium, silicon, sodium, magnesium, cobalt, nickel, molybdenum, vanadium, strontium, and selenium.

29. The amino acid chelate composition of claim 27, wherein the amino acid ligand includes an amino acid selected from the group consisting of arginine, asparagine, cystine, glutamine, histidine, lysine, ornithine, and tryptophan, including dipeptides, tripeptides, and tetrapeptides thereof.

30. The amino acid chelate composition of claim 27, wherein the second nitrogen-containing compound is a proteinate.

31. The amino acid chelate composition of claim 27, wherein the second nitrogen-containing compound is urea.

32. The amino acid chelate composition of claim 27, wherein the second nitrogen-containing compound is a nitrate.

33. The amino acid chelate composition of claim 27, wherein the second nitrogen-containing compound is carnitine.

34. The amino acid chelate composition of claim 27, wherein the second nitrogen-containing compound is creatine.

35. The amino acid chelate composition of claim 27, wherein the second nitrogen-containing compound is glucosamine.

36. The amino acid chelate composition of claim 27, wherein the second nitrogen-containing compound is chondroitin.

37. The amino acid chelate composition of claim 27, wherein the second nitrogen-containing compound is chitosan.

38. The amino acid chelate composition of claim 27, wherein the second nitrogen-containing compound is a nitrogen-containing botanical.

39. The amino acid chelate composition of claim 27, wherein the second nitrogen-containing compound includes at least two nitrogen atoms.

40. The amino acid chelate composition of claim 27, further comprising a glandular substance.

41. The amino acid chelate composition of claim 27, wherein the amino acid ligand includes at least three nitrogen atoms.

42. The amino acid chelate composition of claim 27, wherein the amino acid ligand includes at least four nitrogen atoms.

43. The amino acid chelate composition of claim 27, wherein the amino acid chelate has an amino acid to metal ratio from about 1:1 to about 4:1.

44. The amino acid chelate composition of claim 27, wherein the nitrogen content from the amino acid chelate and second nitrogen-containing compound is from about 5 wt % to about 35 wt %.

45. The amino acid chelate composition of claim 27, wherein the animal is a mammal.

46. The amino acid chelate composition of claim 27, wherein the animal is a human.

47. The amino acid chelate composition of claim 27, wherein the animal is a fowl.

48. The amino acid chelate composition of claim 27, wherein the animal is a fish.

49. The amino acid chelate composition of claim 27, wherein the animal is a crustacean.

50. The amino acid chelate composition of claim 27, further comprising a metal bisglycinate chelate.

51. The amino acid chelate composition of claim 27, in oral dosage form.

52. The amino acid chelate composition of claim 27, furthering including a nitrogen-containing salt including a second metal and an anion.

53. The amino acid chelate composition of claim 52, wherein the second metal is independently selected from the group consisting of copper, zinc, manganese, iron, chromium, calcium, sodium, potassium, silicon, magnesium, cobalt, nickel, molybdenum, vanadium, strontium, and selenium.

54. The amino acid chelate composition of claim 52, wherein the metal and the second metal are the same.

55. The amino acid chelate composition of claim 52, wherein the metal and the second metal are different.

56. The amino acid chelate composition of claim 52, wherein the anion is selected from the group consisting of nitrates, amino acid sulfates, and ureates.

57. A method of increasing a metabolic activity in an animal tissue, comprising administering an amino acid chelate composition including an amino acid chelate having a multi-nitrogen-containing amino acid ligand and a metal to an animal in an amount sufficient to i) raise the nitrogen and the metal concentration within the tissue, ii) retain metal content in the tissue for a greater period of time compared to when the metal is delivered as a compound with less nitrogen, and iii) enhance metabolic activity of the tissue.

58. The method of claim 57, wherein the multi-nitrogen-containing amino acid ligand includes an amino acid selected from the group consisting of arginine, asparagine, cystine, glutamine, histidine, lysine, ornithine, and tryptophan, including dipeptides, tripeptides, and tetrapeptides thereof.

59. The method of claim 57, wherein the multi-nitrogen-containing amino acid ligand includes at least three nitrogen atoms.

60. The method of claim 57, wherein the multi-nitrogen-containing amino acid ligand includes at least four nitrogen atoms.

61. The method of claim 57, wherein the nitrogen content from the amino acid chelate is from about 5 wt % to about 35 wt %.

62. The method of claim 57, wherein the animal is a mammal.

63. The method of claim 57, wherein the animal is a human.

64. The method of claim 57, wherein the animal is a fowl.

65. The method of claim 57, wherein the animal is a fish.

66. The method of claim 57, wherein the animal is a crustacean.

67. The method of claim 57, wherein the metabolic activity is milk production, enhanced growth, enhanced fertility, reduced morbidity, reduced tissue fat, or enhanced feed conversion.

68. The method of claim 57, wherein the step of administering is by oral administration.

69. The method of claim 57, including co-administering a second amino acid chelate that is different than the amino acid chelate, said second amino acid chelate including a second metal and a second amino acid ligand.

70. The method of claim 69, wherein the second amino acid ligand includes an amino acid selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

71. The method of claim 69, wherein the metal and the second metal are independently selected from the group consisting copper, zinc, manganese, iron, chromium, calcium, potassium, sodium, silicon, magnesium, cobalt, nickel, molybdenum, vanadium, strontium, and selenium.

72. The method of claim 69, wherein the metal and the second metal are the same, and the multi-nitrogen-containing amino acid ligand and the second amino acid ligand are different.

73. The method of claim 69, wherein the metal and the second metal are different, and the multi-nitrogen-containing amino acid ligand and the second amino acid ligand are different.

74. The method of claim 69, wherein the metal and the second metal are different, and the multi-nitrogen-containing amino acid ligand and the second amino acid ligand are the same.

75. The method of claim 69, wherein the second amino acid ligand includes at least two nitrogen atoms.

76. The method of claim 69, wherein the amino acid chelate and the second amino acid chelate each have an amino acid ligand to metal ratio from about 1:1 to about 4:1.

77. The method of claim 69, wherein the amino acid chelate composition has an amino acid chelate to second amino acid chelate weight ratio from about 10:1 to about 1:10.

78. The method of claim 57, including co-administering a nitrogen-containing non-chelate salt including a second metal and an anion.

79. The method of claim 69, wherein the metal and the second metal are independently selected from the group consisting of copper, zinc, manganese, iron, chromium, calcium, potassium, sodium, silicon, magnesium, cobalt, nickel, molybdenum, vanadium, strontium, and selenium.

80. The method of claim 69, wherein the metal and the second metal are the same.

81. The method of claim 69, wherein the metal and the second metal are different.

82. The method of claim 69, wherein the anion is selected from the group consisting of nitrates, amino acid sulfates, and ureates.

83. The method of claim 57, including co-administering a second nitrogen-containing compound.

84. The method of claim 83, wherein the second nitrogen-containing compound includes at least two nitrogen atoms.

85. The method of claim 83, wherein the second nitrogen-containing compound is selected from the group consisting of nitrogen-containing salts, proteinates, urea, nitrates, carnitine, creatine, glucosamine, chondroitin, chitosan, nitrogen-containing botanicals, glandular substances, and combinations thereof.

86. The method of claim 83, wherein the nitrogen content from the amino acid chelate and second nitrogen-containing compound is from about 5 wt % to about 35 wt %.

87. The method of claim 83, formulated for mammal delivery.

88. The method of claim 83, formulated for human delivery.

89. The method of claim 83, formulated for fowl delivery.

90. The method of claim 83, formulated for fish delivery.

91. The method of claim 83, formulated for crustacean delivery.

92. The method of claim 83, wherein the step of administering is by a formulation selected from the group consisting of oral, injection, powder, tablet, capsule, gel, liquid, or paste.

93. The method of claim 92, wherein the formulation is oral or injection.
